SEQUENCE LISTING (10 sequences)

SEQ ID NO: 1 - DNA, 46 bp, Artificial Sequence
  mcheery F primer
gggcggatccggtggcagcggcggttcgatggtgagcaagggcgag

SEQ ID NO: 2 - DNA, 29 bp, Artificial Sequence
  mcheery R primer
ggtgctcgagcttgtacagctcgtccatg

SEQ ID NO: 3 - DNA, 31 bp, Artificial Sequence
  ZIF268F primer
tatacatatggaacgcccgtatgcttgccct

SEQ ID NO: 4 - DNA, 50 bp, Artificial Sequence
  ZIF268R primer
accggatccgcccgagccaccgctaccgccgtccttctgtcttaaatgga

SEQ ID NO: 5 - DNA, 29 bp, Artificial Sequence
  oligonucleotide for immobilizing on the substrate(sense strand)
agatcacacacacaccgcccacgccttac

SEQ ID NO: 6 - DNA, 29 bp, Artificial Sequence
  oligonucleotide for immobilizing on the substrate(antisense strand)
gtaaggcgtgggcggtgtgtgtgtgatct

SEQ ID NO: 7 - protein, 15 aa, Artificial Sequence
  peptide linker
GGGGSGGGGSGGGGS

SEQ ID NO: 8 - protein, 6 aa, Artificial Sequence
  peptide linker
GGSGGS

SEQ ID NO: 9 - protein, 11 aa, Artificial Sequence
  peptide linker
GGGGSGGGGGS

SEQ ID NO: 10 - protein, 15 aa, Artificial Sequence
  peptide linker
GGSGGSGGSGGSGGS

(19) United States
(12) Patent Application Publication
(10) Pub. No.: US 2012/0064510 A1
(43) Pub. Date: Mar. 15, 2012

(54) KIT INCLUDING TARGET SEQUENCE-BINDING PROTEIN AND METHOD OF DETECTING TARGET NUCLEIC ACID BY USING THE KIT

(75) Inventors: Joo-won RHEE, Yongin-si (KR); Su-hyeon KIM, Seoul (KR)

(73) Assignee: SAMSUNG ELECTRONICS CO., LTD., Suwon-si (KR)

(21) Appl. No.: 13/224,540

(22) Filed: Sep. 2, 2011

(30) Foreign Application Priority Data
Sep. 10, 2010 (KR) 10-2010-0088990

(51) Int. Cl. G01N 33/53; G01N 21/64; G01N 21/75; C12Q 1/70; C12Q 1/68

(52) U.S. Cl. 435/5; 436/501; 435/6.19; 435/6.12

(57) ABSTRACT

A kit including a target sequence-binding protein and a method of detecting a target nucleic acid by using the kit that may ensure efficient detection of the target nucleic acid in a biological sample are disclosed.

### CROSS-REFERENCE TO RELATED APPLICATIONS

This application claims priority to Korean Patent Application No. 10-2010-0088990, filed on Sep. 10, 2010, and all the benefits accruing therefrom under 35 U.S.C. §119, the disclosure of which is incorporated herein by reference.

### BACKGROUND

1. Field

The present disclosure relates to a kit including a target nucleic acid sequence-binding protein, and a method of detecting a target nucleic acid by using the kit.

2. Description of the Related Art

Zinc finger proteins are a class of sequence-specific DNA binding proteins that include a polypeptide structural motif called a zinc finger domain (or motif) that may bind specifically to various target DNA sequences. Zinc finger domains may be used to construct various recombinant polypeptides that specifically recognize particular base sequences for detection. Zinc finger domains have a very strong binding force to DNA, and can be coupled to various fluorescence reporter proteins, which fluoresce at various wavelengths. Thus, specifically detecting target nucleic acids by using recombinant zinc finger proteins has been tried.

Nucleic acid sequence determination has applications in single nucleotide polymorphism (SNP) discrimination, and pathogenic infection, viral infection, and genetic disease diagnosis. Existing nucleic acid diagnostic assay techniques mostly involve amplification. Major drawbacks of amplification-based nucleic acid diagnostic assays are the likelihood of a false positive response caused by contaminants during the amplification and a highly probable error in predicting the concentration of the original unamplified target nucleic acid from amplification-based nucleic acid assay results.

Therefore, there is demand for probes that are specific to a target nucleic acid and so highly sensitive that they do not require amplification for detection and diagnosis.

### SUMMARY

Provided is a kit for determining a nucleotide sequence of a target nucleic acid and a method for determining a nucleotide sequence of a target nucleic acid.

In an embodiment, the kit includes a target sequence-binding protein including an amino acid sequence that specifically binds to a target nucleic acid sequence; and a detectable tag linked to the target sequence-binding protein. In some embodiments, the detectable tag is mCherry fluorescent protein.

In an embodiment, the kit includes at least two target sequence-binding proteins which are labeled with different detectable tags and include amino acid sequences specifically binding to different target nucleotide sequence

In an embodiment, a method of detecting a target nucleic acid includes contacting a nucleic acid with a target sequence-binding protein which is labeled with a detectable tag and which includes an amino acid sequence that specifically binds to a target nucleotide sequence, wherein the detectable tag is mCherry fluorescent protein; and detecting a signal from the detectable tag indicating binding between the target sequence-binding protein and the target nucleotide sequence.

### BRIEF DESCRIPTION OF THE DRAWINGS

These and/or other aspects of the invention will become apparent and more readily appreciated from the following description of the various exemplary embodiments, taken in conjunction with the accompanying drawings of which:

FIG. 1 is a schematic diagram of a polynucleotide encoding a fusion protein of a target sequence-binding protein linked to a detectable tag, showing the various domains in the fusion protein and locations of restriction sites used in construction of the expression vector for the fusion protein;

FIG. 2 is a schematic diagram of an expression vector pET21b-Zif268-DsRed including the polynucleotide shown in FIG. 1;

FIG. 3 is a sodium dodecyl sulfate-polyacrylamide gel electrophoresis (SDS-PAGE) image of the zif268-mCherry fusion protein expressed from the expression vector of FIG. 2; Lane 1 to lane 3 are purified protein solutions which were loaded 2 ul, 1 ul, and 0.5 ul, respectively.

FIG. 4A presents two EM-CCD images from a single-molecule bleaching analysis experiment for the zif268-mCherry fusion protein;

FIG. 4B is a graph of fluorescence intensity from the detectable tag of the zif268-mCherry fusion protein as a function of time (in seconds), showing loss of fluorescence with time, x-axis is “time” and “oligo” means fluorescence intensity of Cy5 which labeled the target nucleic acid; and

FIG. 5 is an EM-CCD image of a sample with a target nucleic acid labeled with Cy5 and the zif268-mCherry fusion protein, a graph of fluorescence intensity of the target nucleic acid (“oligo”) or the zif268-mCherry fusion protein as a function of time (in seconds) and a graph of fluorescent resonance energy transfer (FRET) between the target nucleic acid and the zif268-mCherry fusion protein measured as a function of time (in seconds).

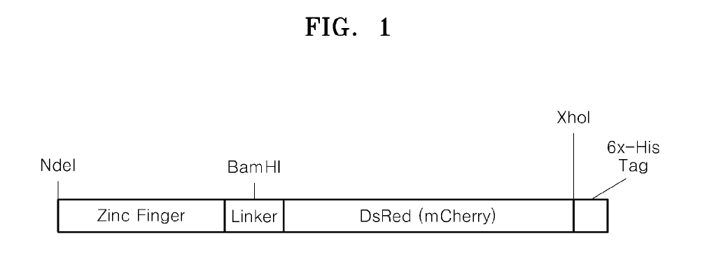

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description.

Disclosed herein is a kit for determining a nucleotide sequence of a target nucleic acid. In an embodiment, the kit includes at least two target sequence-binding proteins which are labeled with different detectable tags and include amino acid sequences that specifically bind to different target nucleic acid sequences.

Disclosed herein are a method and an apparatus for determining a nucleotide sequence of a target nucleic acid.

One embodiment provides a kit for detecting a target nucleic acid, the kit including a target sequence-binding protein and a detectable tag linked to the target sequence-binding protein, wherein the target sequence-binding protein includes an amino acid sequence that specifically binds to a target nucleic acid sequence.

As used herein, the term “nucleic acid” refers to a polymer of nucleotides. The nucleic acid may include deoxyribonucleic acid (DNA; gDNA and cDNA) and/or ribonucleic acid (RNA), peptide nucleic acid (PNA), or locked nucleic acid (LNA). Nucleotides, which are the basic building blocks of nucleic acids, include not only natural nucleotides such as deoxyribonucleotide and ribonucleotide, but also artificial analogues including a modified sugar or base. Natural deoxyribonucleotides include four types of bases: adenine (A), thymine (T), guanine (G), and cytosine (C). Ribonucleotides generally include a base that is C, A G, or uracil (U). The abbreviations, A, T or U, C, and G are used herein to describe either the base or the nucleotide in a nucleic acid sequence, according to context.

As used herein, the term “target sequence-binding protein” refers to a kind of protein capable of recognizing and binding to a specific nucleotide sequence (specific recognition sequence) of a target nucleic acid. The amino acid sequence in the target sequence-binding protein which specifically recognizes the specific nucleotide recognition sequence of a target nucleic acid may include a nucleic acid-binding motif. The target sequence-binding protein may include at least one nucleic acid-binding motif. In some embodiments the amino acid sequence specifically binding to the specific nucleotide recognition sequence may include at least one nucleic acid-binding motif selected from the group consisting of a zinc finger motif, a helix-turn-helix motif, a helix-loop-helix motif, a leucine zipper motif, the nucleic acid-binding motif of a restriction endonuclease, and combinations thereof.

In some embodiments the amino acid sequence may include a zinc finger motif. The target sequence-binding protein may include one to five zinc finger motifs, and in some embodiments, may include one to three zinc finger motifs.

The zinc finger motif may have any of the various zinc finger amino acid backbone structures known in the art, and in some embodiments, may be selected from the group consisting of a “Cys₂His₂” zinc finger, “Cys₄” zinc finger, “His₄” zinc finger, “His₃Cys” zinc finger, “Cys₃X” zinc finger, “His₃X” zinc finger, “Cys₂X₂” zinc finger, “His₂X₂” zinc finger (wherein X is a zinc-ligating amino acid) and combinations thereof, which are non-limiting examples of zinc finger motif backbone structures.

The target sequence-binding protein comprising at least one zinc finger motif may specifically recognize and bind to a specific nucleotide recognition sequence. In some embodiments the specific nucleotide recognition sequence may have a nucleotide length of about 3 to about 21, and in some embodiments, may have a nucleotide length of about 6 to about 18. In some embodiments, a Cys₂His₂ zinc finger motif may include an α-helical seven amino acid sequence that specifically recognizes a three nucleotide long sequence. Zinc finger motifs may specifically recognize different nucleotide sequences. Nucleotide sequences specifically recognized by certain amino acid sequences of zinc finger motifs can be obtained using, for example, the internet-based program, Zinc Finger Tools (Mandell J G, Barbas C F 3rd. Zinc Finger Tools: custom DNA-binding domains for transcription factors and nucleases. Nucleic Acids Res. 2006 Jul. 1; 34 (Web Server issue):W516-23.).

In some embodiments the zinc finger motif may be a wild-type zinc finger motif, a mutant type zinc finger motif, or a combination thereof. A mutant zinc finger motif may include about 1 to about 5 amino acid residues substituting for those of a wild-type zinc finger motif, and in some embodiments, may include about 2 to about 4 such substituted amino acid residues. These substituted amino acid residues may specifically bind to the nucleic acid.

A library of zinc finger motifs capable of specifically recognizing and binding to specific nucleotide sequences may be constructed by random mutation of an initial zinc finger motif on the gene level. For example, a phage display method by which a zinc finger motif library is displayed on a phage surface, a yeast one-hybrid method, a bacterial two-hybrid method, or a cell-free translation may be used to screen zinc finger motifs.

In some embodiments the target sequence-binding protein may be linked with a detectable tag.

As used herein, the term “detectable tag” refers to a moiety used to specifically detect a molecule or substance including the moiety from among the same type of molecules or substances without the moiety. The moiety can be an atom or a molecule. In some embodiments the detectable tag may be a colored bead, an antigen determinant, an enzyme, hybridizable nucleic acid, a chromophore, a fluorescent material, a phosphorescent material, an electrically detectable molecule, a molecule providing modified fluorescence-polarization or modified light-diffusion, a quantum dot, or the like. In addition, the detectable tag may be a radioactive isotope such as P³² or S³⁵, a chemiluminescent compound, labeled binding protein, a heavy metal atom, a spectroscopic marker such as a dye, or a magnetic label. The dye may be a quinoline dye, a triarylmethane dye, phthalene, an azo dye, or a cyanine dye, but is not limited thereto. Suitable fluorescent materials may include Alexa Fluor 350, Alexa Fluor 430, Alexa Fluor 488, Alexa Fluor 532, Alexa Fluor 546, Alexa Fluor 568, Alexa Fluor 594, Alexa Fluor 633, Alexa Fluor 647, Alexa Fluor 660, Alexa Fluor 680, Cy2, Cy3.18, Cy3.5, Cy3, Cy5.18, Cy5.5, Cy5, Cy7, mCherry, Oregon Green, Oregon Green 488-X, Oregon Green, Oregon Green 488, Oregon Green 500, Oregon Green 514, SYTO 11, SYTO 12, SYTO 13, SYTO 14, SYTO 15, SYTO 16, SYTO 17, SYTO 18, SYTO 20, SYTO 21, SYTO 22, SYTO 23, SYTO 24, SYTO 25, SYTO 40, SYTO 41, SYTO 42, SYTO 43, SYTO 44, SYTO 45, SYTO 59, SYTO 60, SYTO 61, SYTO 62, SYTO 63, SYTO 64, SYTO 80, SYTO 81, SYTO 82, SYTO 83, SYTO 84, SYTO 85, SYTOX Blue, SYTOX Green, SYTOX Orange, SYBR Green YO-PRO-1, YO-PRO-3, YOYO-1, YOYO-3, and thiazole orange. In some embodiments the detectable tag may be contained in the target sequence-binding protein to specifically detect binding of the target sequence-binding protein to the specific nucleotide recognition sequence.

In some embodiments the target sequence-binding protein and the detectable tag may be coupled by a linker. In some embodiments the linker may attach to the N-terminus or C-terminus of the target sequence-binding protein. The linker may be a non-peptide linker or a peptide linker.

The non-peptide linker may be any of various compounds that may be used as linkers in the art. A suitable linker may be selected based on the type of a functional group in the protein (polypeptide) that binds to the target sequence. In some embodiments the linker may be an alkyl linker or an amino linker. The alkyl linker may be a branched or non-branched, cyclic or acylic, substituted or unsubstituted, saturated or unsaturated, chiral, achiral or racemic mixture. In some embodiments the alkyl linker may have 2 to 18 carbon atoms. Other suitable alkyl linkers may include at least one functional group selected from among hydroxy, amino, thiol, thioether, ether, amide, thioamide, ester, urea, and thioether. The alkyl linker may be a 1-propanol linker, a 1,2-propanediol linker, a 1,2,3-propanetriol linker, a 1,3-propandiol linker, a triethylene glycol hexaethylene glycol linker, a polyethylene glycol linker (for example, [—O—CH₂—CH₂—]ₙ (n=1-9)), a methyl linker, an ethyl linker, a propyl linker, a butyl linker, or a hexyl linker.

The peptide linker may be any of various linkers that are widely used in the art, and in some embodiments, may be a linker including a plurality of amino acid residues. The peptide linker may allow the target sequence-binding protein and the detectable tag (for example, a fluorescent protein) in a fusion protein to be spaced apart from each other by a distance that is sufficient enough to allow each polypeptide domain to fold into appropriate secondary and tertiary structures. For example, the peptide linker may include Gly, Asn and Ser residues, and in some other embodiments, may include neutral amino acid residues, such as Thr and Ala. Amino acid sequences suitable for the peptide linker are known in the art. Suitable amino acid sequences may include (Gly₄-Ser)₃(SEQ ID NO: 7), (Gly₂-Ser)₂(SEQ ID NO: 8), and Gly₄-Ser-Gly₅-Ser (SEQ ID NO: 9). The linker may be unnecessary, and in some embodiments, may have various lengths, as long as it does not affect functions of the target sequence-binding protein and the detectable tag.

In some embodiments, the kit for detecting a target nucleic acid may include a reagent for stabilizing the target sequence-binding protein. For example, the kit may include a buffer solution known in the art. In some embodiments, the kit may be manufactured to have a plurality of separate packages or compartments.

Another embodiment provides a recombinant vector library including: a polynucleotide sequence coding for a fusion protein including a target sequence-binding protein and a detectable tag linked to the target sequence-binding protein; and a promoter operatively linked to the polynucleotide sequence.

Herein, the term “vector” refers to a vector used to express a target gene in a host cell. The vector may include a plasmid vector, a cosmid vector, and a virus vector, such as a bacteriophage vector, an adenovirus vector, a retrovirus vector, and an adeno-associated virus vector. Suitable recombinant vectors may be constructed by manipulating plasmids that are widely used in the art, such as pSC101, pGV1106, pACYC177, ColE1, pKT230, pME290, pBR322, pUC8/9, pUC6, pBD9, pHC79, pIJ61, pLAFR1, pHV14, pGEX series, pET series, and pUC19; phages, such as λgt4λB, λ-Charon, λΔz1, and M13; or viruses, such as SV40.

In the recombinant vector, the sequence of the polynucleotide coding for the fusion protein may be operatively linked to a promoter. As used herein, the term “operatively linked” indicates a functional binding of a nucleotide expression control sequence (e.g., a promoter sequence) to another nucleotide sequence, wherein the nucleotide expression control sequence may control transcription and/or translation of the other nucleotide sequence thereby.

The recombinant vector may be an expression vector that stably expresses the fusion protein in a host cell. The expression vector may be a conventional vector that is used in the art to express an exogenous protein in plants, animals, or microorganisms. The recombinant vector may be constructed using various methods known in the art.

The recombinant vector may be constructed for use in a prokaryotic cell or a eukaryotic cell as host. For example, if the recombinant vector is an expression vector for a prokaryotic host cell, the vector may include a promoter capable of initiating transcription, such as p_{L}^{λ} promoter, trp promoter, lac promoter, tac promoter, and T7 promoter, a ribosome-binding site to initiate translation, and a transcription/translation termination sequence. If a eukaryotic cell is used as the host cell, the vector should include an origin of replication operating in the eukaryotic cell, which may be a f1 replication origin, a SV40 replication origin, a pMB1 replication origin, an adeno replication origin, an AAV replication origin, or a BBV replication origin, but is not limited thereto. The promoter used in the recombinant vector may be a promoter derived from a genome of a mammal cell (for example, a metalthionine promoter) or a promoter derived from a virus of a mammal cell (for example, an adenovirus anaphase promoter, a vaccinia virus 7.5K promoter, a SV40 promoter, a cytomegalo virus promoter, or a tk promoter of HSV) and may include a polyadenylated sequence as a transcription termination sequence.

Another embodiment provides a cell transformed by the recombinant vector.

Any host cell known in the art to enable stable and continuous cloning or expression of the recombinant vector may be used. Suitable prokaryotic host cells may include E. coli JM109, E. coli BL21, E. coli RR1, E. coli LE392, E. coli B, E. coli X 1776, E. coli W3110, Bacillus species strains such as Bacillus subtillis or Bacillus thuringiensis, intestinal bacteria and strains such as Salmonella typhymurum, Serratia marcescens, and various Pseudomonas species. Suitable eukaryotic host cells to be transformed may include yeasts, such as Saccharomyce cerevisiae, insect cells, plant cells, and animal cells, for example, Chinese hamster ovary (CHO), W138, BHK, COS-7, 293, HepG2, 3T3, RIN, and MDCK cell lines.

The polynucleotide or the recombinant vector including the polynucleotide may be transferred into a host cell by using a known transfer method. Suitable transfer methods may be chosen according to the host cell. Suitable transfer methods for prokaryotic host cells may include a method using CaCl₂ or electroporation. Suitable transfer methods for eukaryotic host cells may include microinjection, calcium phosphate precipitation, electroporation, liposome-mediated transfection, and gene bombardment. However, any suitable transfer method may be used.

The transformed host cell may be screened using a phenotype expressed by a selectable marker, and known methods. For example, if the selectable marker is a gene that is resistant to a specific antibiotic, a transformed host cell may be easily screened by being cultured in a medium containing the antibiotic.

According to another embodiment of the present invention, a method of detecting a target nucleic acid sequence includes: contacting a target nucleic acid with a target sequence-binding protein which is labeled with a detectable tag and includes an amino acid sequence that specifically binds to a specific nucleotide recognition sequence; and detecting a signal from the detectable tag indicating formation of a complex between the target sequence-binding protein and the specific nucleotide recognition sequence. In an embodiment, the method further comprises identifying that the specific nucleotide recognition sequence is present in the target nucleic acid. In some embodiments the contacting the nucleic acid may be performed after a biological sample is acquired. The biological sample may be any sample containing nucleic acid. Nonlimiting examples of biological samples are blood, tear drops, buccal swabs, saliva, viruses, and microorganisms.

In some embodiments the contacting may be achieved by mixing the target-sequence binding protein, and a biological sample or the nucleic acid extracted from the biological sample in a liquid medium. The liquid medium may be any buffer solution known in the art to maintain stabilities of the target sequence-binding protein and the target nucleic acid and to be permit specific binding of the target sequence-binding protein with its specific nucleotide recognition sequence. The contacting allows a nucleic acid binding motif of the target sequence-binding protein to approach the target nucleic acid and specifically bind to its specific nucleotide recognition sequence if it is present in the target nucleic acid. The contacting may be followed by washing away any target sequence-binding protein that remains unbound to the target nucleic acid.

The target nucleic acid may be double-stranded. The target nucleic acid may have any of various lengths depending on the length of the nucleic acid extracted from a biological sample. The target nucleic acid may be prepared having various lengths by using a known method in the art. For example, the target nucleic acid may have a length of about 100 bp to about 10 Mb, and in some embodiments, may have a length of about 1 kb to about 1 Mb.

In some embodiments the target nucleic acid may further include a detectable tag. The target nucleic acid may be labeled with the detectable tag when being prepared. Suitable detectable tags are the same as those described above.

In some embodiments the method may further include detecting a signal from the detectable tag linked to the target nucleic acid.

Detecting the signal generated from the detectable tag can be performed by using a suitable detector. In some embodiments examples of signals generated from the detectable tag include a signal selected from the group consisting of a magnetic signal, an electric signal, a light emitting signal such as a fluorescent or Raman signal, a diffusion signal, and a radioactive signal. Examples of the detection signal are the same as described above in conjunction with the detectable tag.

In regard to the embodiment comprising detecting a signal from the detectable tag linked to the target sequence-binding protein, the target nucleic acid to be brought into contact with the target sequence-binding protein may be a biological sample, i.e., not an artificially synthesized polynucleotide. If the target nucleic acid is an isolated polynucleotide, the isolated polynucleotide may also be labeled with a detectable tag to detect a signal by using fluorescent resonance energy transfer (FRET) between the detectable tag bound to the target nucleic acid and the detectable tag labeling the target sequence-binding protein and thus determine the presence of complex formation between the target nucleic acid and the target sequence-binding protein. “Isolated,” when used to describe the various polypeptides, fusion proteins, or polynucleotides disclosed herein, means a polypeptide, fusion protein, or polynucleotide that has been identified and separated and/or recovered from a component of its natural environment. The term also embraces recombinant polynucleotides and polypeptides and chemically synthesized polynucleotides and polypeptides.

The present invention will be described in further detail with reference to the following examples. These examples are for illustrative purposes only and are not intended to limit the scope of the invention.

### Example 1

### Preparation of Target Sequence-Binding Protein Linked with Detectable Tag

Constructing a vector to express a fusion protein of a target sequence-binding protein linked with a detectable tag (the mCherry fluorescent protein, “mCherry”) and purifying the fusion protein expressed using the vector are described below.

In order to synthesize the target sequence-binding fusion protein, a polynucleotide fragment coding for part of a (Gly₂Ser)₅ (SEQ ID NO: 10) linker and the fluorescent protein, mCherry, was obtained by polymerase chain reaction (PCR). The amplification of the polynucleotide fragment was performed using pmCherry (Clontech, cat. no. 632522) as template, and the primers mCherry F primer (SEQ ID NO. 1), coding for part of the (Gly₂Ser)₅ linker and including a nucleotide sequence cleavable by BamHI, and mCherry R primer (SEQ ID NO. 2) including a nucleotide sequence cleavable by XhoI. The amplification was performed using a GENEAMP® PCR System 9700 (Applied Biosystems) under the following PCR conditions: at 95° C. for 5 minutes; at 95° C. for 20 seconds; repeated 30 times at 68° C. for 2 minutes; at 68° C. for 5 minutes; and cooled to 4° C. The resulting PCR product was purified using a QIAQUICK® Multiwell PCR Purification kit (Qiagen) according to the manufacturer's protocol and was used in subsequent steps. The amplified PCR product was cleaved with BamHI and XhoI restriction enzymes and inserted into a pET21b (Novagen) vector, which was cleaved with the same two restriction enzymes, to construct a vector, pET21b-DsRed.

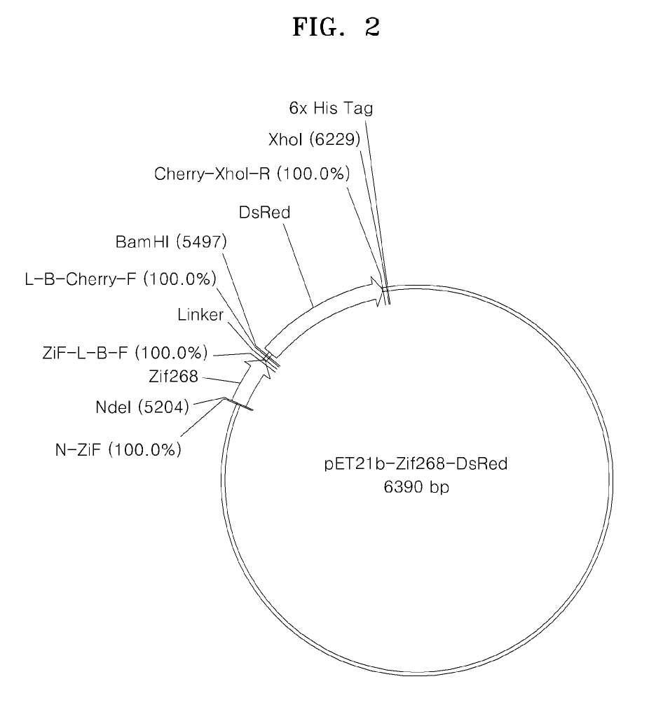

Amplification was also performed using the plasmid pCSZif268 (Kim and Pabo, 1998, PNAS, 95:2812-2817) as template and the primers, ZIF268F primer (SEQ ID NO. 3) including a nucleotide sequence cleavable with NdeI and ZIF268R primer (SEQ ID NO. 4) coding for part of the (Gly₂Ser)₅ linker and including a nucleotide sequence cleavable with BamHI, to obtain the target sequence-binding protein. The amplification was performed using a GENEAMP®PCR System 9700 (Applied Biosystems) under the following PCR conditions: at 95° C. for 5 minutes; at 95° C. for 20 seconds; repeated 30 times at 68° C. for 2 minutes; at 68° C. for 5 minutes; and cooled to 4° C. The resulting PCR product was purified using a QIAQUICK®Multiwell PCR Purification kit (Qiagen) according to the manufacturer's protocol and was used in subsequent steps. The amplified PCR product was cleaved with BamHI and NdeI restriction enzymes and inserted into the pET21b-DsRed vector, cleaved with the same two restriction enzymes, to construct the vector, a pET21b-Zif268-DsRed (see FIG. 2).

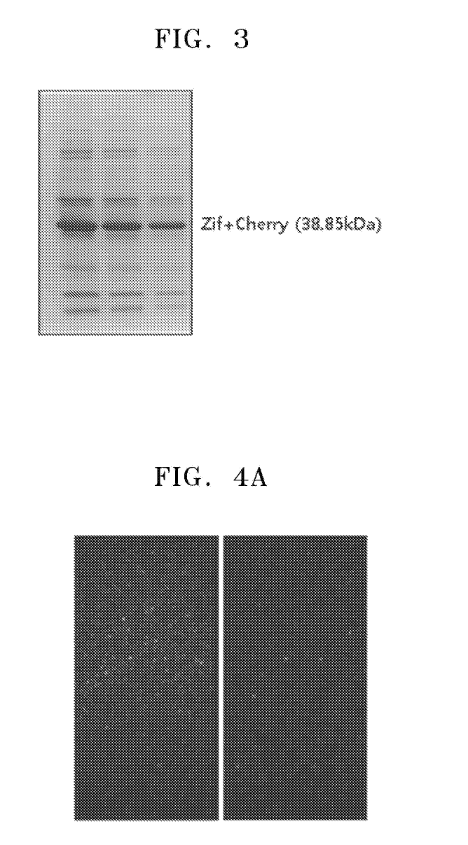

In order to use the pET21b-Zif268-DsRed vector to over-express the encoded fusion protein, the pET21b-Zif268-DsRed vector was transformed into E. coli BL21 (DE3). Luria Broth (LB) liquid medium to which 50 μg/ml of ampicillin was added was used as the culture medium for the transformed E. coli BL21 (DE3). Isopropyl-β-d-thiogalactopyranoside (IPTG) was added to the culture medium at 0.5 mM when the optical density (O.D., absorbance) of the culture reached a value of 0.5 at a 600-nm wavelength, and then the transformed E. coli BL21 (DE3) was further cultured at about 25° C. for about 16 hours. After sonication in a 25 mM Tris-HCl buffer solution (pH 8.0), the cell culture was centrifuged (at 10,000×g) to obtain a supernatant. The supernatant was loaded on a Ni²⁺-NTA superflow column (Qiagen) equilibrated with the buffer solution, and then washed with a wash buffer solution volume five times higher than the volume of the column. Then, protein was eluted from the column with an elution buffer solution (25 mM Tris-HCl (pH 8.0), 2.5 mM β-mercaptoethanol, 125 mM imidazole, and 150 mM NaCl). Fractions including the fusion protein were collected and filtered using AMICON® Ultra-15 Centrifugal Filters (Millipore) to remove salts from the fractions. Then, the desalted fractions were concentrated. The concentrated mCherry-linker-ZFP fusion protein (“zif-mCherry”) was dissolved and stored in storage solution A (25 mM Tris-HCl (pH 8.0), 2.5 mM β-mercaptoethanol, 125 mM imidazole, 150 mM NaCl, and 50% glycerol) or storage solution B (20 mM Tris-HCl (pH 7.5), 1 mM DTT, 100 mM NaCl, and 50% glycerol). The concentration of the purified protein was quantified using bovine serum albumin (BSA) as the standard material. FIG. 3 shows that the fusion protein had a molecular weight of 38.85 kDa and was separated with a high purity.

### Example 2

### Single-Molecule Detection by Bleaching

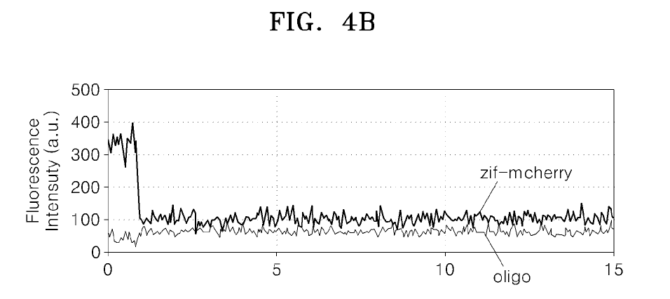

A 10 μM solution of the fusion protein of Example 1 in 10 mM Tris-HCl, 1 mM EDTA (TE) buffer (pH 7.4) was randomly adsorbed on a microfluidic device manufactured using glass-quartz, and was bleached by irradiation with light at a wavelength of 532 nm. As shown in FIG. 4A, in an oxygen scavenger-free solution, most of the spots emitting fluorescence in the left panel of FIG. 4A lost fluorescence within one second, as shown in the right panel of FIG. 4A. FIG. 4B is a graph showing fluorescence intensity as a function of time, showing loss of fluorescence with time for the zif-mCherry fusion protein. Referring to FIG. 4B, the loss of fluorescence occurred at once in one second, which indicates that the fusion protein may be detected on a single-molecule basis.

### Example 3

### Manufacture of Microfluidic Device for Immobilization of Fusion Protein-Specific DNA

A microfluidic device was manufactured as described below, for immobilizing a polynucleotide including a nucleotide sequence to which the fusion protein of Example 1 could specifically bind on a surface of a substrate (cover glass).

### Example 3-1

### Cover Glass Washing

Twenty pieces of cover glass were prepared and five pieces were put into each of four respective staining jars, taking care not to contaminate the surfaces of each cover glass. Ethanol was added to each staining jar, which was then sonicated for about 30 minutes. Each staining jar was washed with distilled water four to five times, and 5001 ml of a 1M KOH solution was added thereto, which was then sonicated for about 30 minutes and washed again with distilled water four to five times. These processes were repeated once more. Then, acetone was added to each staining jar to remove moisture, and was sonicated for about 10 minutes. Then, the surface of each staining jar was wiped with tissue to remove moisture, and was then washed with acetone twice. Then, the staining jars were filled with acetone.

### Example 3-2

### Silanization

400 ml of acetone was mixed with 8 ml of silane to prepare a 2% (v/v) silane solution. The acetone was removed from each staining jar, and the silane solution was added thereto. The staining jars were shaken to facilitate silane-SiO₂ binding, and were then left on a table for about 2 minutes. Then, 1.5 l of distilled water was poured into each staining jar to an appropriate height at an appropriate rate to terminate silanization, and each staining jar was then washed with distilled water two to three times. Then, distilled water was added thereto. The cover glasses were then taken out of each staining jar, placed on aluminum foil, and dried in an oven at 110° C. for about 30 minutes.

### Example 3-3

### PEGylation

Methylated polyethylene glycol (mPEG)-biotin-N-hydroxysuccinimide (NHS) and mPEG-NHS that had been stored at −20° C. were left at room temperature for about 25 minutes. Separately, a 0.1M NaHCO₃ buffer solution (pH 8.3) was prepared. One to two milligrams (mg) of mPEG-biotin-NHS and 100 mg of mPEG-NHS were mixed with 500 μl of the NaHCO₃ buffer solution, which was then vigorously shaken. Then, an additional 500 μl of the NaHCO₃ buffer solution was added, and vigorously shaken. Ten additional pieces of cover glass were prepared for use as spacers, different from the silanized cover glasses that were taken from the oven. Initially, ten of the silanized cover glasses, taken from the oven, were placed on an appropriate box, and spacer cover glasses were placed thereon, to bridge edges of every two adjacent cover glasses. 100 μl of the mPEG-biotin solution was dropped onto a clean surface of each silanized cover glass, which was then covered with another silanized cover glass prepared in Example 3-2. After being left at room temperature for about 3 hours, the upper cover glasses were carefully separated from the lower ones, which were subsequently used in microfluidic devices. The removed upper cover glasses were completely cleaned with distilled water, and then with nitrogen gas to remove the remaining water. The cleaned cover glasses were placed in a clean box, which was placed in a vacuum chamber for later use.

### Example 4

### Detection of Specific DNA Recognition Sequence of Fusion Protein by Using FRET

Polynucleotides (SEQ ID NOs. 5 & 6) including complementary sequences to form the duplex specific recognition sequence (target nucleotide sequence) of the target sequence-binding fusion protein were hybridized, and immobilized at 0.1 pM concentration on a functionalized cover glass, manufactured in Example 3-3, for about 10 minutes, such that one strand of the duplex polynucleotide to be fixed to the cover glass had been reacted with the biotin-NHS functional group on the cover glass, and the other strand is functionalized with Cy5.

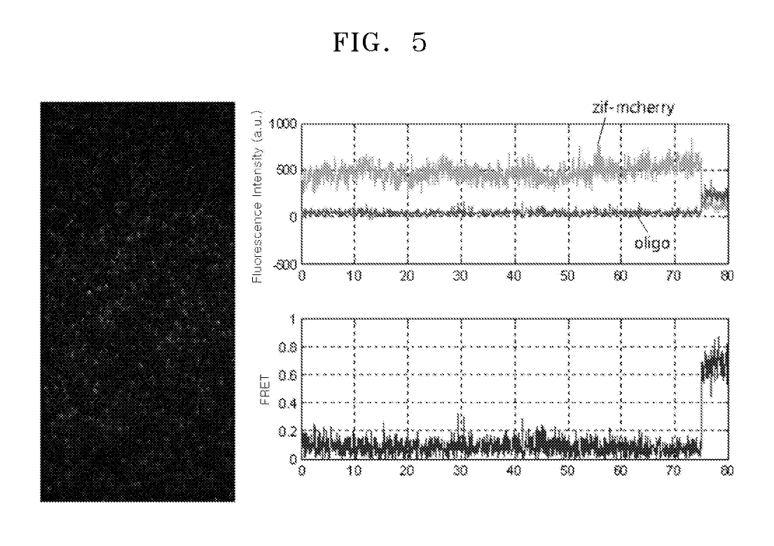

Then, 6 nM of the fusion protein synthesized in Example 1 was slowly flowed over the microfluidic device, which was then irradiated with light and the intensities of emitted fluorescence signals at wavelengths corresponding to mCherry and Cy5 was detected using an Electron Multiplying Charge Coupled Device (EM-CCD) camera (HAMAMATSU). The excitation wavelength of the light was 532 nm. The distance between the Cy5 label and mCherry in the fusion protein was about 8 nm, which was marginal for a fluorescent resonance energy transfer (FRET) reaction. As shown in FIG. 5, FRET signals were detected from the Cy5 channel after about 70 seconds from the beginning of fluorescence measurement.

Using the methods described in the above exemplary embodiments, binding of the fusion protein to a specific target DNA sequence may be induced, and the presence of the specific target DNA sequence bound with the fusion protein may be specifically detected with high sensitivity.

As described above, according to the one or more of the above embodiments of the present invention, a kit including a target sequence-binding protein and a method of detecting a target nucleic acid by using the kit may ensure efficient detection of the target nucleic acid in a biological sample.

The terminology used herein is for the purpose of describing particular embodiments only and is not intended to be limiting of the invention. The terms “a” and “an” do not denote a limitation of quantity, but rather denote the presence of at least one of the referenced item. The terms “comprising”, “having”, “including”, and “containing” are to be construed as open-ended terms (i.e. meaning “including, but not limited to”).

Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable.

All methods described herein can be performed in a suitable order unless otherwise indicated herein or otherwise clearly contradicted by context. No language in the specification should be construed as indicating any non-claimed element as essential to the practice of the invention as used herein.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

It should be understood that the exemplary embodiments described herein should be considered in a descriptive sense only and not for purposes of limitation. Descriptions of features or aspects within each embodiment should typically be considered as available for other similar features or aspects in other embodiments.

What is claimed is:

1. A kit for determining nucleotide sequence of a target nucleic acid comprising
at least two target sequence-binding proteins,
wherein each target sequence-binding protein comprises
an amino acid sequence that specifically binds to a target nucleic acid sequence; and
a detectable tag linked to the target sequence-binding protein,
wherein each target sequence-binding protein specifically binds a target nucleotide sequence different from that of any other target sequence-binding protein in the kit and is labeled with a different detectable tag from that of any other target sequence-binding protein in the kit.

2. A kit for detecting a target nucleic acid, the kit comprising:
a target sequence-binding protein comprising
an amino acid sequence that specifically binds to a target nucleic acid sequence; and
a detectable tag linked to the target sequence-binding protein, wherein the detectable tag is mCherry fluorescent protein.

3. The kit of claim 2, wherein the target nucleotide sequence comprises about 6 to about 8 arbitrary nucleotides.

4. The kit of claim 2, wherein the amino acid sequence comprises at least one nucleic acid-binding motif selected from the group consisting of a zinc finger motif, a helix-turn-helix motif, a helix-loop-helix motif, a leucine zipper motif, a nucleic acid-binding motif of restriction endonuclease, and combinations thereof.

5. The kit of claim 2, wherein the amino acid sequence comprises about 1 to about 5 zinc finger motifs.

6. The kit of claim 2, wherein the amino acid sequence comprises the zinc finger domain of ZIF268.

7. The kit of claim 2, wherein the target sequence-binding protein further comprises a linker connecting the amino acid sequence that specifically binds to the target nucleic acid sequence and the detectable tag.

8. A method of detecting a target nucleic acid, the method comprising:
contacting a nucleic acid with a target sequence-binding protein which is labeled with a detectable tag and includes an amino acid sequence that specifically binds to a target nucleotide sequence, wherein the detectable tag is mCherry fluorescent protein; and
detecting a signal from the detectable tag indicating binding between the target sequence-binding protein and the target nucleotide sequence.

9. The method of claim 8, further comprising before the contacting acquiring a biological sample comprising the nucleic acid.

10. The method of claim 9, wherein the biological sample is blood, tear drops, saliva, a buccal swab, a virus, or a microorganism.

11. The method of claim 8, wherein the nucleic acid is double-stranded.

12. The method of claim 8, wherein the nucleic acid further comprises a second detectable tag.

13. The method of claim 12, wherein the second detectable tag comprises at least one selected from the group consisting of a colored bead, a chromophore, a fluorescent material, a phosphorescent material, an electrically detectable molecule, a molecule providing modified fluorescence-polarization or modified light-diffusion, and a quantum dot.

14. The method of claim 8, wherein the nucleic acid has a length of about 100 bp to about 10 Mb.

15. The method of claim 12, wherein detecting the signal comprises
detecting a signal produced by fluorescent resonance energy transfer (FRET) between the second detectable tag of the nucleic acid and the detectable tag labeling the target sequence-binding protein.

16. The kit of claim 2, further comprising
at least one additional target sequence-binding protein,
wherein each target sequence-binding protein specifically binds a target nucleotide sequence different from that of any other target sequence-binding protein in the kit and is labeled with a different detectable tag from that of any other target sequence-binding protein in the kit.
